(19) United States
(12) Patent Application Publication
(10) Pub. No.: US 2016/0270802 A1
(43) Pub. Date: Sep. 22, 2016

(54) GUIDING ELEMENT FOR SPINAL DRILLING OPERATION AND GUIDING ASSEMBLY COMPRISING THE SAME

(71) Applicant: NATIONAL CHENG KUNG UNIVERSITY, Tainan City (TW)

(72) Inventors: Jing-Jing FANG, Tainan City (TW); Ruey-Mo LIN, Tainan City (TW); Sheng-Yi FANG, Pingtung City (TW); Ya-Wen SHIH, Tainan (TW)

(21) Appl. No.: 15/170,323

(22) Filed: Jun. 1, 2016

Related U.S. Application Data
(63) Continuation-in-part of application No. 13/429,099, filed on Mar. 23, 2012, now abandoned.

(30) Foreign Application Priority Data
Mar. 25, 2011 (TW) 100110288
Mar. 13, 2012 (TW) 101108536

(51) Int. Cl. A61B 17/17; A61B 17/16

(52) U.S. Cl. CPC A61B 17/1757; A61B 17/1671; A61B 17/1637

(57) ABSTRACT

The present invention discloses a guiding element and a guiding assembly for spinal drilling operation. The guiding element is configured to be dispose at a vertebra which comprises a lamina. The guiding element comprises a main body, two connection parts, and two locating parts. The main body has two stand portions and each of the stand portion comprises a clamping part. Each of the connection parts comprises a first end and a second end. The two first ends extends away from each other. Each locating part connects to each stand portion through each connection part. Each clamping part comprises two hook portions. Each side, which is configured to be in contact against the vertebra, of the hook portions has a curvature corresponding to the surface of the lamina, so that the main body is configured to be disposed at the lamina and across the vertebra through the hook portions.

### CROSS REFERENCE TO RELATED APPLICATIONS

This Non-provisional application is a Continuation-In-Part of U.S. application Ser. No. 13/429,099 filed on Mar. 23, 2012 which claims priority under 35 U.S.C. §119(a) on Patent Application No(s). 100110288 filed in Taiwan, Republic of China on Mar. 25, 2011, and Application No(s). 101108536 filed in Taiwan, Republic of China on Mar. 13, 2012, the entire contents of which are hereby incorporated by reference.

### BACKGROUND OF THE INVENTION

1. Field of Invention

The present invention relates to a guiding element and a guiding assembly for drilling operation, and in particular, to a guiding element and a guiding assembly for spinal drilling operation.

2. Related Art

Spine is such an important element in human body because it not only constructs the main frame of skeleton but has the function of protecting the inside spinal cord, which is the spindle of human nerve system. The nerves protrude outwardly from the inside of the vertebrae, and then extend forwardly, laterally, and vertically to the visceral organs as well as the extremity of human body. In other words, the vertebrae are the origin point of the neural network system of human body.

Vertebrae include cervical vertebrae, thoracic vertebrae, and lumbar vertebrae. Nerves come from the spinal cord and go out through the intervals between the vertebral bodies. Unfortunately, if the deformation of any vertebral body of the vertebrae occurs, the nerve is possibly pressed, which may indirectly affect the organ, muscle and gland body connected to the nerve. This undesired deformation can result a huge injury to human health.

The reasons of the vertebral dislocation include the spondylolisthesis caused by vertebral degeneration, injury (e.g. dislocation or fracture), infection, tumor, and the likes. The major therapy for the vertebrae deformation is to implant a transpedicular screw on the pedicle of a vertebra (or vertebral body). The rigidity of the transpedicular screw can push the vertebra back the normal position and fix it, so that the relative positions of the vertebrae are more stable so as to avoid the compression and wearing of nerves and release the pain of patient.

In the recent years, the spinal surgery has been progressively developed. Especially, the transpedicular screw further provides a revolutionary progress, and it can be applied to not only the easier lumbar vertebrae operation but also the more difficult cervical and thoracic vertebrae operation. However, the implantation of transpedicular screw still exists a certain possibility of complication, especially for the cervical and thoracic vertebrae operation. This unpredictable risk of these surgeries really stops many patients.

Since the shape of each vertebra may different in different bodies, it has been an important issue of the surgery to precisely implant the transpedicular screw into the center of the pedicle of the targeted vertebra.

Accordingly, the location methods applied in the spinal drilling operation have been studied. The conventional location methods are mainly divided three types. The first type includes the steps of preoperation CT scan and intraoperative registration. The second type is to use an adjustable operative accessory for measuring before the surgery and then perform the operation with the well-adjusted operative accessory. The third type is to produce a customized guiding element for a specific patient to perform the operation.

However, although the first type can achieve a good precision, it spends a lot time on registration. Besides, the operator must confirm the angles and positions again and again during the operation. These complex and extra procedures can interfere with the operation. Regarding to the second type, the precise guiding result is merely achieved because the errors can be caused by manual operation and, more important, the position of the target vertebra may different before and during the surgery.

The third method need more time and cost on the preparation for customization design before the surgery, but it has the advantages of high precision and easy operation. As shown in FIG. 4, the convention method is to utilize an auxiliary stand 51 during the operation with the customized guiding element 54. The auxiliary stand 51 includes two support legs 511, each of which is configured with a guide 513. The auxiliary stand 51 is designed with a V-shaped knife-edge for standing on the spinous process and transverse process of the vertebra. During the operation, the operator places the auxiliary stand 51 to contact against the spinous process and transverse process of the vertebra so as to locate the auxiliary stand 51, and then uses the guide 513 to guide the drill 514 for successfully performing the reaming procedure on the vertebra.

Although the above method can reduce the error in the operation, it has a problem in keeping the auxiliary stand at the desired position. In details, the auxiliary stand is stood on the surface of the vertebra through the bottom of the support legs only, and the body of the auxiliary stand does not configured with any other mechanism for contacting or fastening to the vertebra. Thus, the auxiliary stand is easily swayed even the operator carefully holds it. In addition, the guide of the auxiliary stand can provide the reference for the drilling location, but it may lose stability during the drilling/reaming, and even worse, the auxiliary stand may misalign with the center of the pedicle. Thus, the location stability during the reaming procedure is still insufficient.

In the U.S. Patent Application Publication No. US20130053854, a patient-specific alignment guide is disclosed. Please refer to FIG. 5, the alignment guide 61 is only suitable for the operation of the inferior end of the spine (i.e., the sacroiliac portion of the spine). In addition, the alignment guide 61 can only be firmly disposed at the targeted bone with other components. The operator has to fix some parts of the alignment guide 61, such as the locking portions 621, 622, 623, 624, and 625, with the iliac crest of the patient at first through the screws, then the alignment guide 61 can be disposed. In other words, the alignment guide 61 cannot be firmly fixed on the targeted place without other component. Therefore, the difficulty and the complexity of the operation are increased.

In the U.S. Patent Application Publication No. US20110319745, a pedicle screw guide is disclosed. Please refer to FIG. 6, the pedicle screw guide 71 can only be firmly disposed at the target place when all three portions (i.e., the two cylindrical columns 713 and the longitudinal cavity 720) contact with the vertebra of the patient. Therefore, during the drilling process, the operator cannot observe the drilling situation and the location where the pin actually drills into the vertebra. The precision of spinal drilling operation may therefore be jeopardized.

Therefore, it is an important subject of the present invention to provide a guiding tool for spinal drilling operation that is easily operated and has the specificity for patient's vertebra and high location stability and precision, thereby improving the efficiency and application of the customized guiding element on the location and drilling/reaming issues during spinal drilling operation.

### SUMMARY OF THE INVENTION

In view of the foregoing subject, an objective of the present invention is to provide a guiding element and a guiding assembly for spinal drilling operation. When compared with the conventional auxiliary fixing frame, the guiding assembly is easily operated and has the specificity for patient's vertebra and high location stability and precision, so that it can improve the efficiency and application of the customized guiding element on the location and drilling/reaming issues during spinal drilling operation.

To achieve the above objectives, the present invention discloses a guiding element for spinal drilling operation. The guiding element is configured to be disposed at a vertebra, and the vertebrae comprises a lamina. The guiding element comprises a main body, two connection parts, and two locating parts. The main body has two stand portions and each of the stand portion comprises a clamping part. The each of the connection parts comprises a first end and a second end. Each second end connects with one of the stand portions. Each connection part extends in a direction from each second end to each first end, and the two first ends extends away from each other. Each locating part connects to each stand portion through each connection part. Each clamping part comprises two hook portions, and each clamping part faces toward each other. The main body is configured to be disposed at the lamina of the vertebra through the hook portions of the clamping parts, and each side, which is configured to be in contact against the vertebra, of the hook portions has a curvature corresponding to the surface of the lamina, and the sides, which are configured to be in contact against the vertebra, of the each two hook portions of the same clamping part are continuous to one side of the same clamping part being configured to be in contact against the vertebra are continuous, so that the main body is configured to be disposed at the lamina and across the vertebra through the hook portions.

In one embodiment, the main body further has a hand-held portion connecting to the stand portion.

In one embodiment, the hand-held portion has a through hole.

In one embodiment, each connection part forms an angle with each stand portion, and the angle is larger than 90 degrees.

In one embodiment, each locating part is dangling from the vertebra through each connection part.

In one embodiment, the vertebra is a cervical vertebra, thoracic vertebra, or a lumbar vertebra.

In one embodiment, the two clamping part extends toward and connects to each other and forms an integral part.

To achieve the above objectives, the present invention also discloses a guiding assembly. The guiding assembly comprises a guiding element for spinal drilling operation, at least an auxiliary element, at least a k-pin, and at least a cannular driller. The guiding element is configured to be dispose at a vertebra, and the vertebrae comprises a lamina. The guiding element comprises a main body, two connection parts, and two locating parts. The main body has two stand portions and each of the stand portion comprises a clamping part. Each of the connection parts comprises a first end and a second end. Each second end connects with one of the stand portions. Each connection part extends in a direction from each second end to each first end, and the two first ends extends away from each other. Each locating part connects to each stand portion through each connection part. Each clamping part comprises two hook portions, and each clamping part faces toward each other. The main body is configured to be disposed at the lamina of the vertebra through the hook portions of the clamping parts, and each side, which is configured to be in contact against the vertebra, of the hook portions has a curvature corresponding to the surface of the lamina, and the sides, which are configured to be in contact against the vertebra, of the each two hook portions of the same clamping part are continuous to one side of the same clamping part being configured to be in contact against the vertebra are continuous, so that the main body is configured to be disposed at the lamina and across the vertebra through the hook portions. The auxiliary element is mounted on one of the locating part. The k-pin is mounted on the auxiliary element, and the cannular driller is mounted on the k-pin and having a holding part and a drilling part.

In one embodiment, the main body further has a hand-held portion connecting to the stand portion.

In one embodiment, the hand-held portion has a through hole.

In one embodiment, each connection part forms an angle with each stand portion, and the angle is larger than 90 degrees.

In one embodiment, each locating part is dangling from the vertebra through each connection part.

In one embodiment, the vertebra is a cervical vertebra, thoracic vertebra, or a lumbar vertebra.

In one embodiment, the two clamping part extends toward and connects to each other and forms an integral part.

In one embodiment, the auxiliary element has a through hole along a longitudinal direction, and the k-pin passes through the through hole in the step of mounting the k-pin on the auxiliary element.

As mentioned above, the present invention provides a guiding element and a guiding assembly for spinal drilling operation. The guiding assembly is composed of several elements, including the guiding element, auxiliary element, k-pin and cannular driller, so that the stability and precision of the reaming procedure during the spinal drilling operation can be enhanced.

Moreover, the specific functions of the above elements can help the spinal drilling operation. Regarding to the guiding element, since it has simple structure and is suitable for customization, the demand of the patient can be satisfied. Besides, the guiding element has a portion fitting the vertebra. In other words, the clamping part has the hook portion which is configured to face or bend toward the lamina. In addition, each of the one side of the hook portion being configured to be in contact against the vertebra has a curvature corresponding to the surface of the lamina, so that the main body is configured to be disposed at the spinal process and across the vertebra through the hook portions. Accordingly, the connection between the guiding element and the vertebra can be further improved. In addition, the auxiliary element and the cannular driller are configured to cooperate with the locating part. The auxiliary element can assist the location of the k-pin and prevent the non-stability in the conventional hand-hold procedure. The cannular driller can perform the reaming process through the locating part, thereby increasing the precision and decreasing the safety of spinal drilling operation.

Compared with the conventional art, the present invention still remains the conventional advantages of customizable and easy operation, and can further provide higher location stability and precision due to the structural property of the guiding element. Furthermore, through the guiding element, as well as the guiding assembly for spinal drilling operation, an auxiliary element is used for facilitating and adjusting the penetrating position of the k-pin. This configuration can precisely match the hole drilled by the cannular driller with the targeted pedicle, so that the surgeons can precisely drill into the center of the pedicle of the targeted vertebra.

Preferably, the guiding element of the present invention can have customized design based on the surface angle of the targeted vertebra (e.g. cervical, thoracic or lumber vertebra) of the patient. Accordingly, when the clamping part is pressed so as to push the guiding element, the guiding element can be still fixed on the targeted vertebra firmly, thereby increasing the precision and safety of the spinal drilling operation.

### BRIEF DESCRIPTION OF THE DRAWINGS

The invention will become more fully understood from the detailed description and accompanying drawings, which are given for illustration only, and thus are not limitative of the present invention, and wherein:

FIGS. 1A, and 1C to 1E are schematic diagrams showing different elements of a guiding assembly for spinal drilling operation according to a preferred embodiment of the present invention;

FIG. 1B is a schematic diagram showing the guiding assembly of FIG. 1A disposed at a targeted vertebra according to a preferred embodiment of the present invention;

FIG. 2A is a schematic diagram showing another guiding element of the guiding assembly for spinal drilling operation according to another preferred embodiment of the present invention;

FIG. 2B is a schematic diagram showing the guiding element of FIG. 2A disposed at a targeted vertebra according to the preferred embodiment of the present invention;

FIG. 2C are schematic diagrams showing the guiding element of FIG. 2A for spinal drilling operation according to another preferred embodiment of the present invention;

FIG. 3 is a flow chart of a method for spinal drilling operation according to the preferred embodiment of the present invention;

FIG. 4 is a schematic diagram showing a conventional customized guiding element and auxiliary stand for spinal drilling operation;

FIG. 5 is a schematic diagram showing another conventional customized guiding element and auxiliary stand for spinal drilling operation; and

FIG. 6 is a schematic diagram showing still another conventional customized guiding element and auxiliary stand for spinal drilling operation.

### DETAILED DESCRIPTION OF THE INVENTION

The present invention will be apparent from the following detailed description, which proceeds with reference to the accompanying drawings, wherein the same references relate to the same elements. Moreover, the terms proximal, distal, anterior, posterior, medial, or lateral, etc. in the following embodiments are defined according to anatomy posture and indicative direction. Namely, “proximal” refers to facing the head, “distal” refers to facing the foot; “anterior” refers to facing the ventral of the body, “posterior” refers to facing to the dorsal of the body; “medial” refers to facing the central line of the body, “lateral” refers to departing from the central line of the body. Although the following embodiments take human knee for example, they are not limited thereto.

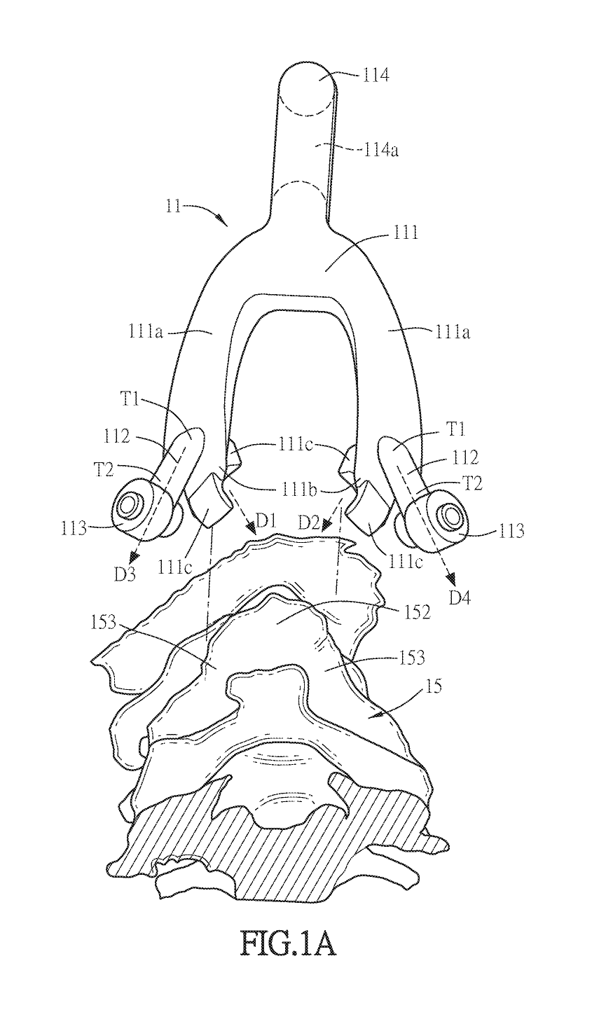

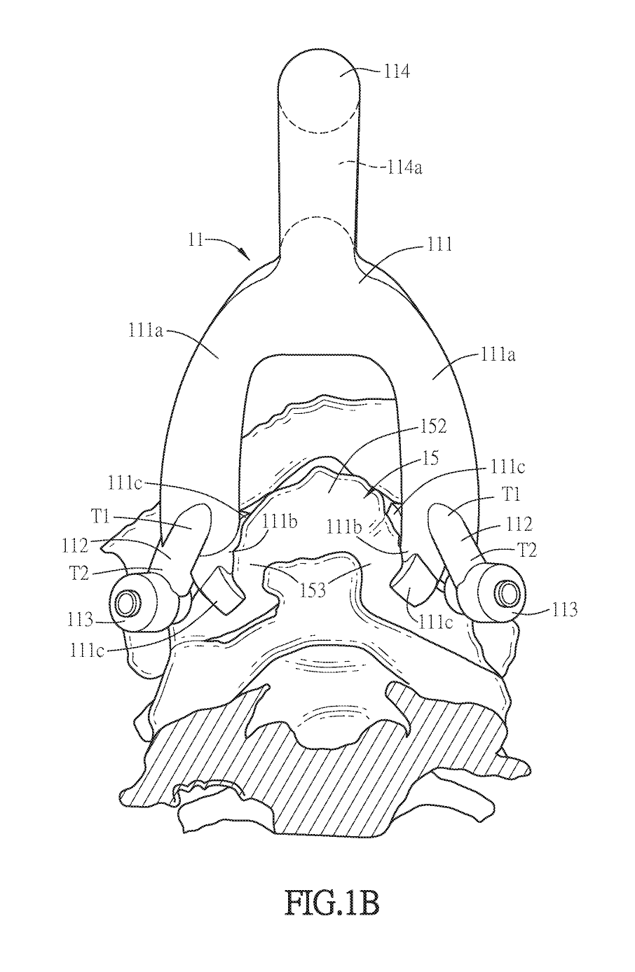

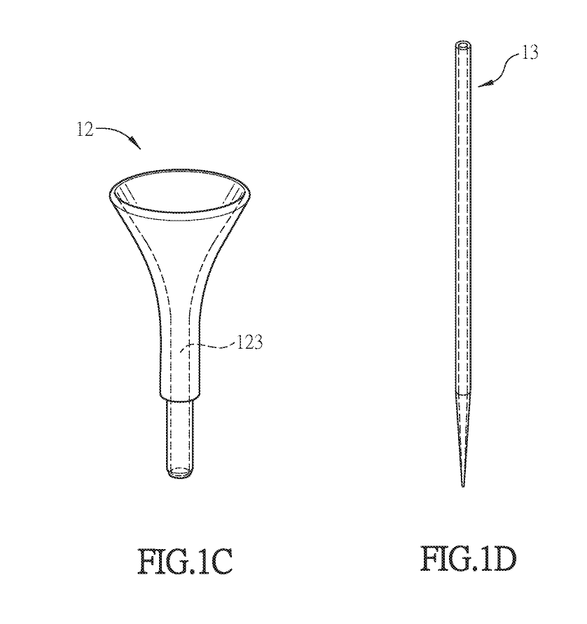

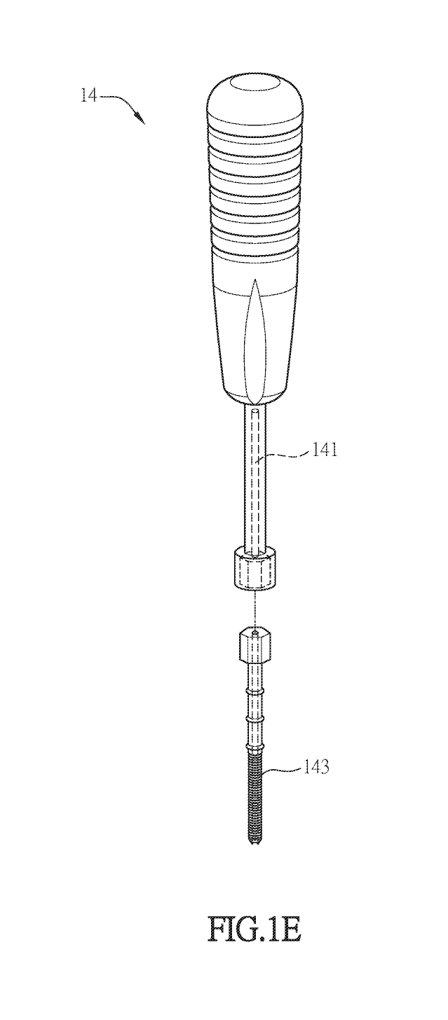

FIGS. 1A and 1C to 1E are schematic diagrams showing different elements of a guiding assembly for spinal drilling operation according to a preferred embodiment of the present invention. Referring to FIGS. 1A to 1E, a guiding assembly for spinal drilling operation includes a guiding element 11, an auxiliary element 12, a k-pin 13, and a cannular driller 14. The structural features of these elements will be described hereinafter.

With reference to FIG. 1A, the guiding element 11 includes a main body 111, two connection parts 112, and two locating parts 113.

The main body 111 has two stand portions 111a. The stand portion 111a is substantially a vertical column. The guiding element 11 is configured to be disposed on a vertebra of a patient. When the guiding element 11 is disposed on the vertebra 15 of the patient, each stand portion 111a extends in a direction from the posterior to the anterior of the patient (i.e., a sagittal direction). The bottom (i.e., the terminal end) of each stand portion 111a connects to the clamping part 111b.

Referring to FIG. 1B, the main body 111 is configured to be disposed at and can be disposed at the lamina 153 of the vertebra 15 by the supporting of the stand portion 111a and the contact of the clamping part 111b. Each clamping part 111b faces toward each other. When disposed at the vertebra, both the two clamping parts extends in a direction from the superior to the inferior of the spinal. Each of the two clamping parts 111b has two hook portions 111c. Each hook portion 111c of the each clamping part 111b extends in a superior-inferior direction (i.e., along the longitudinal direction of the spine of the patient) as shown in FIG. 1A. Each hook portions 111c faces or bends toward to the lamina 153. In addition, the sides, which are configured to be in contact against the vertebra 15, of the each two hook portions 111c of the same clamping part 111b are continuous to one side of the same clamping part 111b being configured to be in contact against the vertebra 15. In the present embodiment, the hook portion 111c of the left clamping part 111b extending toward the inferior direction in FIGS. 1A and 1B and the hook portion 111c of the right clamping part 111b extending toward the inferior direction in FIGS. 1A and 1B are similar to a clip or forceps structure for enhancing the fixing and/or the grabbing strength. In addition, when the guiding element 11 is disposed on the vertebra 15 of a patient, the hook portions 111c grab the lamina of the vertebra 15. Accordingly, the clamping part 111b and the lamina 153 can be connected more firmly by such locking means. Besides, each of the one side, which is configured to be in contact against the vertebra 15, of the hook portions 111c has a curvature corresponding to the surface of the lamina 153 and/or the base of the spinous process 152. Accordingly, the contact between the clamping part 111b and the vertebra 15 can be enhanced and the main body 111 can be disposed at the lamina 153 and across the vertebra 15, so as to prevent the possible swaying the guiding element 11 and increase the stability. In detail, the guiding element 11 is configured to be disposed at the vertebra 15 through the hook portions 111c of the clamping parts 111b. Each of the one side, which is configured to be in contact against the vertebra 15, of the hook portion 111c has a curvature corresponding to the surface of lamina 153 of the vertebra 15 or corresponding to the surface of the base of the spinous process 152, so that the main body 111 can be disposed across the spinal process 152 through the stand portion 111a, the clamping parts 111b, and the hook portions 111c. Besides, since the lamina has a smooth and tilting forwardly area, which easily causes sliding during the operation, the hook portions 111c of the guiding element 11 can contact against lamina 153 of the vertebra 15 having the curvature corresponding to the surface of lamina 153 of the vertebra 15 or to the surface of the base of the spinous process 152. This feature can prevent the guiding element 11 from moving forwardly and thus enhance the stability of the drilling/reaming operation.

In addition, each side of the hook portions 111c being configured to be in contact against the vertebra has a curvature corresponding to the surface of the lamina 153, and the hook portion 111c of the left clamping part 111b extending toward the inferior direction in FIG. 1A and the hook portion 111c of the right clamping part 111b extending toward the inferior direction in FIG. 1A are bending in a direction D1 and D2, respectively. The directions D1 and D2 are substantially extend from the dorsal side to the ventral side of the patient, and the directions D1 and D2 virtually extend toward to each other and virtually cross to each other eventually. Such configuration may contribute to increase the stability of the main body 111 when disposed at and across the vertebra 15.

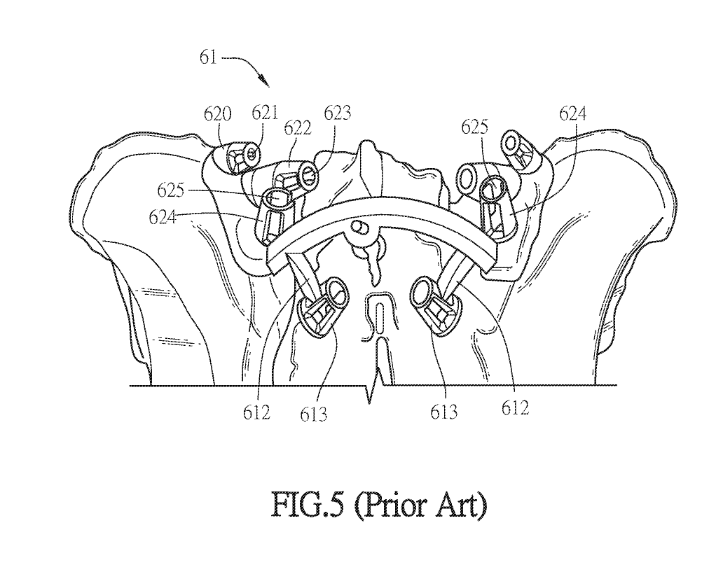

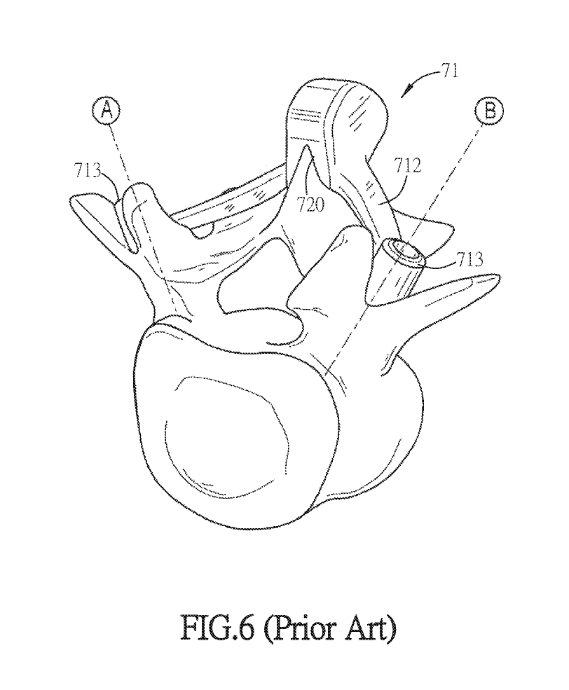

During the fixation process of the conventional alignment guide 61 shown in FIG. 5, the alignment guide 61 can only be firmly disposed at the target place after the locking portions 621, 622, 623, 624, and 625 are fixed with the iliac crest of the patient at first through the screws. However, the guiding element 11 of the present embodiment can be firmly disposed at the lamina 153 of the vertebra 15 and cross the spinous process 152 through the two hook portions 111c of the clamping parts 111b, without other locking means, such as screws. Moreover, the conventional pedicle screw guide 71 shown in FIG. 6 can only be firmly disposed at the target place when all three portions (i.e., the two cylindrical columns 713 and the longitudinal cavity 720) contact with the vertebra of the patient. However, the guiding element 11 of the present embodiment can be firmly disposed at the lamina 153 of the vertebra 15 and cross the spinous process 152 just though two parts contacting with the vertebra 15 (i.e., the two hook portions 111c of the clamping parts 111b).

Please refer to FIG. 1A. Each of the connection parts 112 comprises a first end T1 and a second end T2. Each second end T2 connects with one of the stand portions 111. Both the connection part 112 extend in directions D3 and D4, respectively. The directions D3 and D4 both extend in a direction from each second end T2 to each first end T1. Each locating part 113 connects to each stand portion 111a through each connection part 112. In addition, each stand portion 111a connects to each connection part 112 through each first end T1, and each locating part 113 connects to each connection part 112 through each second end T2. The two first ends T1 extend away from each other.

In other words, when the guiding element 11 is disposed on the vertebra 15, both the directions D3 and D4 extend from the medial to lateral side of the patient. Accordingly, compared to the conventional alignment guide 61, the extending direction of the connection arms 612 is limited or restrained by the configuration of the pelvic bone, both the connection arms 612 are in the lateral-to-medial directions and virtually converge or come close to each other. Therefore, during drilling process by the operator, the operation space is therefore limited. However, the connection parts 112 of the guiding element 11 of the present embodiment extends away from each other and will leave more space at the side of locating parts 113 for operation during the drilling process when the guiding element 11 is disposed on the vertebra 15.

The two locating parts 113 each connect to one side of each of the stand portions 111a through one of the connection parts 112. Preferable, the locating part 113 connects to one side of the stand portion 111a, which is relatively located at the outer side of the spinous process 152. As shown in FIG. 1B, the connection part 112 form an angle θ with the outer side of the stand portion 111a in respect to the vertebra 15. The angle θ is preferably larger than, for example but not limited to, 90 degrees. In practice, the angle θ can be customized according to the shape of the vertebra 15 of the patient and the angle of the drill hole to be formed. If the angle is larger than 90 degrees, the connection part 112 may extend downwards obliquely so as to facilitate the reaming process.

The locating part 113 has a through hole 113a that is provided to confirm the drilling location. Since the guiding element 11 is manufactured based on the information of the vertebra 15 of the patient and the optimum drilling angle and direction estimated by expert or computer, the drilling location can be rapidly figured out by the through hole 113a of the locating part 113 according to the fixing and connecting relations and directions of all elements and parts when the guiding element 11 is provided on the vertebra 15. For example, the projection of the center of the through hole 113a on the vertebra 15 may indicate the center of the location of the drilled hole. Moreover, compared to the conventional the pedicle screw guide 71 shown in FIG. 6, whose cylindrical columns 713 for drilling all close to the vertebra, the two locating parts 113 are both dangling from the vertebra 15 through each connection part 112. In other words, when the guiding element 11 is disposed on the vertebra 15, both of the two locating parts 113 do not directly contact with the vertebra 15 and a gap exists between the locating parts 113 and the vertebra 15. Accordingly, during the drilling process, the operator can observe the drilling situation and the location where the k-pin 13 actually drills into the vertebra 15 through the gap between the locating parts 113 and the vertebra 15, and the precision of spinal drilling operation is therefore increased.

All elements of the above-mentioned guiding element 11 may be integrally formed so as to achieve high stability and precision of the guiding element 11. Of course, the elements of the above-mentioned guiding element 11 may also be individually formed and then assembled by adhering or locking.

In this embodiment, the guiding element 11 is mainly made of resin material such as ABS (SL7580, SL7565 or SL7520, and preferably SL7580). Of course, in other embodiments, the guiding element may be made of any other materials, which has high impact durability, high rigidity and no bio-toxicity, and is easily processed, so that the complexness of the customization of the guiding element can be minimized.

Besides, the guiding element 11 of the embodiment may further include a hand-held portion 114, which is disposed on one side of the stand portion 111a away from the contact portion 111b (upper end in this case). The configuration of the hand-held portion 114 helps the operator to easily hold and operate the guiding element 11. Preferably, the hand-held portion 114 further includes a through hole 114a, which allows the fingers of the operator to pass through, for increasing the utility. In addition, since there are usually more than one targeted vertebrae in the spinal surgery, the hand-held portion 114 can also be used to note the patient name, vertebra location information, and the likes, thereby increasing the safety of the surgery.

FIGS. 1C to 1E are schematic diagrams showing the auxiliary element, k-pin and cannular driller of the guiding assembly for spinal drilling operation according to the embodiment of the present invention. Also reference to FIGS. 1A and 1B, the auxiliary element 12 is mounted on the locating part 113. Preferably, the auxiliary element 12 may further include a hand-held portion 121 for facilitating operations. More preferably, the auxiliary element 12 may further include a through hole 122 to allow the operator to conveniently grab the auxiliary element 12.

The auxiliary element 12 has roughly a pillar shape. The bottom half of the auxiliary element 12 has a diameter and appearance the same as those of the locating part 113. The top half thereof has a slightly larger diameter for limiting the percentage of the locating part 113 mounted therein. With reference to FIGS. 1A to 1E, the k-pin 13 is mounted on the auxiliary element 12, and the cannular driller 14 is mounted on the k-pin 13. The cannular driller 14 includes a holding part 142 and a drilling part 143. In practice, the cannular driller 14 can be any drilling device with a holding part and a threaded drilling head.

Each of the auxiliary element 12, k-pin 13 and cannular driller 14 is integrally formed or assembled with several parts. Besides, each of the auxiliary element 12, k-pin 13 and cannular driller 14 is metal element, so that each of them can have better durability. The auxiliary element 12 has a hollow longitudinal through hole 123, which has the inner diameter and shape the same as those of the k-pin 13, so that the k-pin 13 can penetrate through the through hole 123. The cannular driller 14 also has a hollow part 141 for receiving the k-pin 13, so that the cannular driller 14 can be disposed in the locating part 113 after the hollow part 141 is mounted on the k-pin 13. Accordingly, the cannular driller 14 is guided to the targeted drilling location.

Furthermore, in order to prevent the repulsion issue caused by remaining resin particles when other elements directly contact with the locating part 113, a stainless metal ring 113b is configured at the inner edge of the through hole 113a of the locating part 113. The metal ring 113b is force fitted with the locating part 113, and the contact surface therebetween has a draft pattern design for facilitating the desired mounting and connection.

The guiding assembly for spinal drilling operation of the present invention can be fixed at cervical vertebrae, thoracic vertebrae, or lumbar vertebrae so as to assist the spinal drilling operation. When the guiding assembly is applied to the thoracic vertebrae or to the more difficult cervical or lumbar vertebrae, the opposite inner sides of the hook portion 111c have surface curvatures fitting the appearance of two outer sides of the cervical vertebrae, thoracic vertebrae, or lumbar vertebrae. Thus, the hook portion 111c can totally attach to the laminas of the cervical vertebrae, thoracic vertebrae, or lumbar vertebrae without interfering the joint capsule and ligamentum flavum, thereby improving the surgery safety.

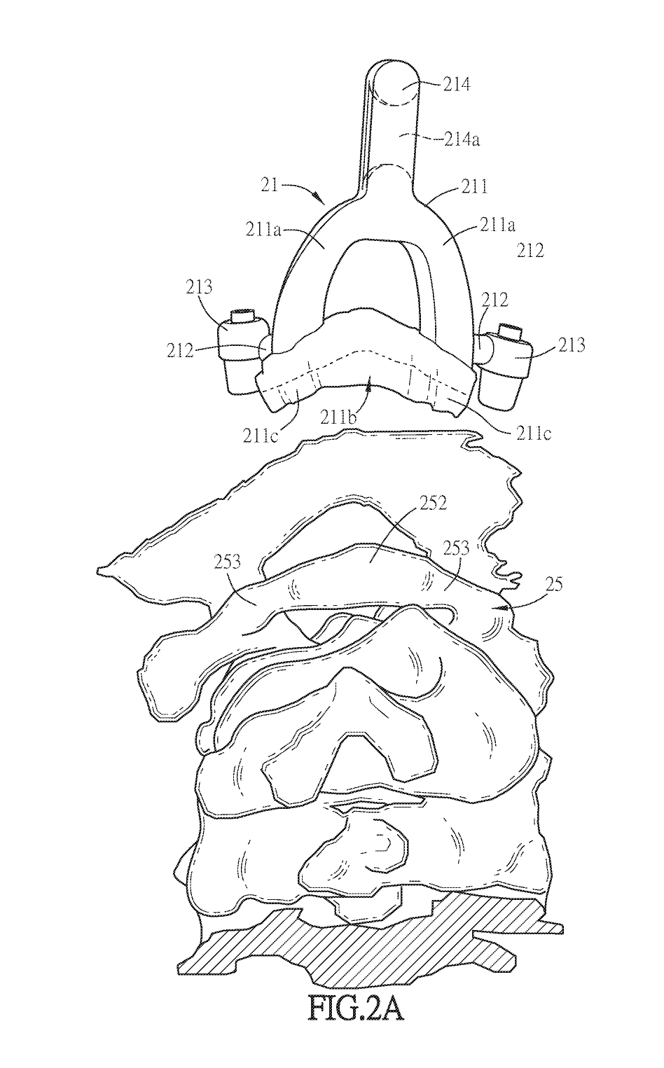

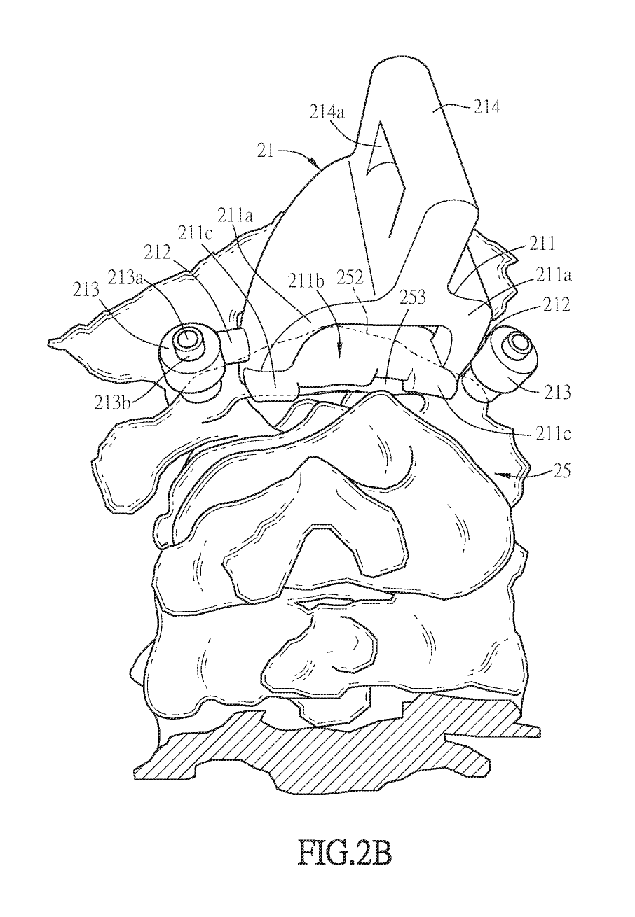

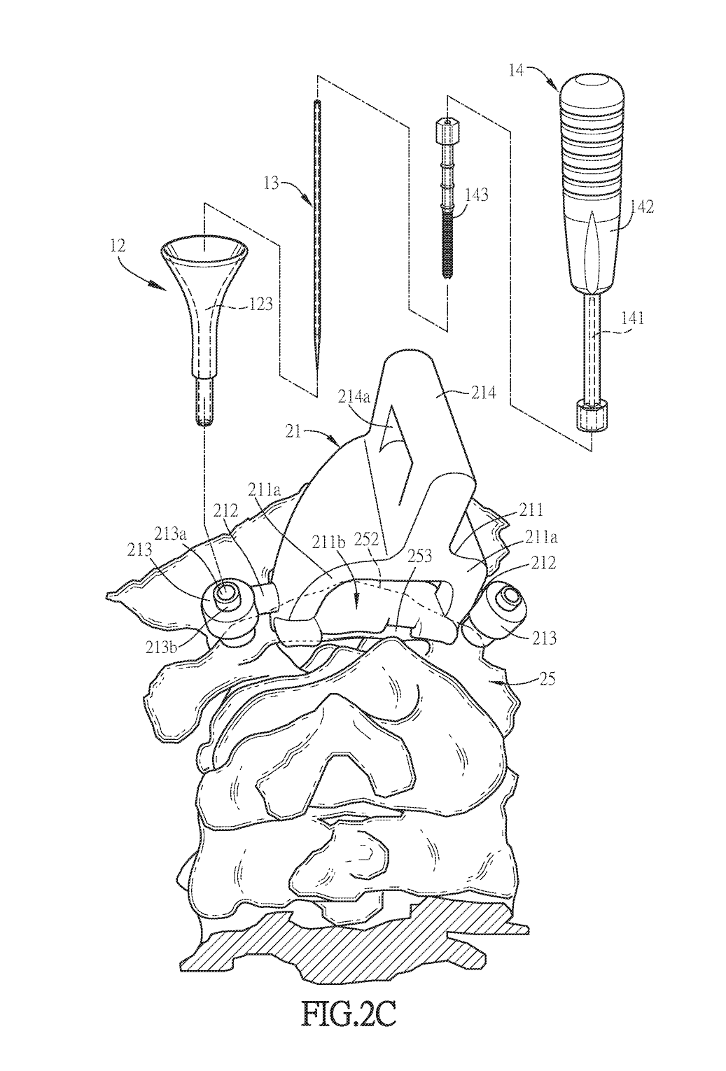

FIGS. 2A and 2B are both schematic diagrams showing another guiding element 21 of the guiding assembly for spinal drilling operation disposed at the vertebra 25 according to the preferred embodiment of the present invention. The structure and composition of the guiding element 21 is mostly the same as those of the previously mentioned guiding element 11, except for that the clamping parts of the guiding element 21 are no longer separated components, but extend toward to each other and form an integral part 211b.

Please refer to FIGS. 2A and 2B. The integral part 211b has functions of both the clamping parts 111b and hook portions 111c. In other words, the one side, which is configured to be in contact against the vertebra 25, of the integral part 211b has a curvature corresponding to the surface of the lamina and/or the base of the spinous process 252. Accordingly, the contact between the integral part 211b and the vertebra 25 can be enhanced and the main body 211 can be disposed at the lamina 253 and across the vertebra 25, so as to prevent the possible swaying the guiding element 21 and increase the stability. In detail, the guiding element 21 is disposed at the vertebra 25 through the hook portions 211c of the integral part 211b. In other words, the one side, which is configured to be in contact against the vertebra 25, of the hook portion 211c has a curvature corresponding to the surface of lamina 253 of the vertebra 25 or corresponding to the surface of the base of the spinous process 252, so that the main body 211 can be disposed across the spinal process 252 through the stand portion 211a, the integral part 211b, and the hook portions 211c. When the guiding element 21 is disposed at the vertebra 25, the main body 211 is across the spinous process 252 of the vertebra 25 as shown in FIG. 2B, and contact with the lamina and/or the base of the spinous process 252 of the vertebra 25 through the integral part 211b which is connected to both the standing portions 211a.

In addition, in the present embodiment, the vertebra 25 is the cervical bone of the cervical vertebrae which is close to the skull, and the thickness of the lamina 253 is thinner than those of the lamina of the inferior cervical vertebrae. The clamping parts of the guiding element 21 is modified and become the integral part 211b to increase the contact area between the guiding element 21 and the lamina 253 when the integral part 211b grabs the lamina 253 and/or the spinous process 252. The guiding element 21 can be also more firmly disposed at the lamina 253 by the configuration of the integral part 211b (like a claw to grab and/or cover the lamina 253) and the drilling stability during the operation is therefore increased.

FIG. 2C is a schematic diagram showing the guiding element 21 as well as other elements of the guiding assembly for spinal drilling operation shown in FIGS. 1C to 1E. To be noted, the center line only shows the relative positions of the elements and is not to limit the present invention. Referring to FIG. 2C, the through hole 213a of the guiding element 21 is force fitted with a metal ring 213b and is disposed on the spinous process of the vertebra. Then, the auxiliary element 12 is mounted to the through hole 213a of the locating part 213, followed by mounting the k-pin 13 through the longitudinal through hole 123 of the auxiliary element 12. After that, the k-pin 13 is inserted into the vertebra along the direction of the longitudinal through hole 123. After the direction and angle of the k-pin 13 is fixed, the x-ray is taken to confirm the direction of the k-pin 13. The auxiliary element 12 is stably removed along the axial direction of the k-pin 13, and the hollow portion 141 of the cannular driller 14 is mounted on the k-pin 13. Thus, the drilling part 143 of the cannular driller 14 can pass through the locating part 313 along the axial direction of the k-pin 13 and then be guided to the targeted drilling position. Finally, the operator can manually rotate the cannular driller 14 to expand the hole for implanting.

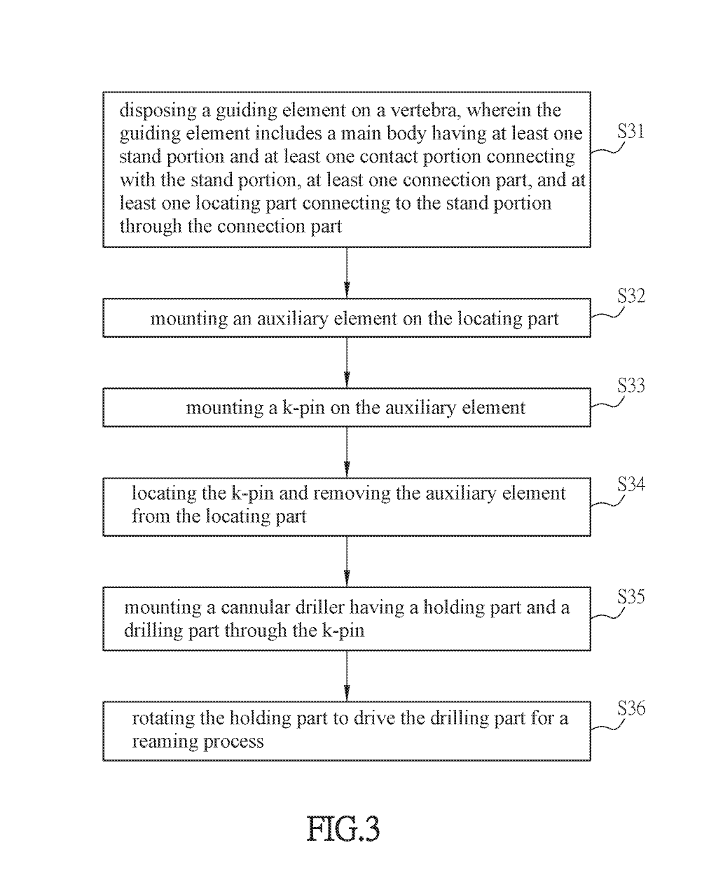

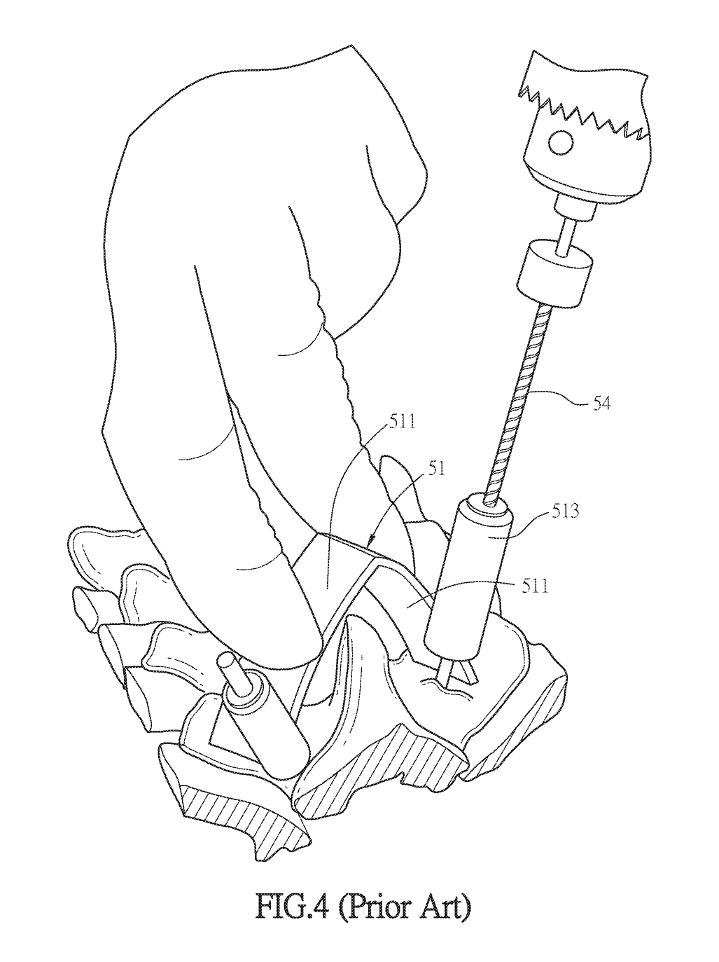

FIG. 3 is a flow chart of a method for spinal drilling operation according to the preferred embodiment of the present invention. Referring to FIG. 3, the method for spinal drilling operation includes the following steps of: disposing a guiding element on a vertebra, wherein the guiding element includes a main body having at least one stand portion and at least one clamping part connecting with the stand portion, at least one connection part, and at least one locating part connecting to the stand portion through the connection part (step S31); mounting an auxiliary element on the locating part (step S32); mounting a k-pin on the auxiliary element (step S33); locating the k-pin and removing the auxiliary element from the locating part (step S34); mounting a cannular driller having a holding part and a drilling part through the k-pin (step S35); and rotating the holding part to drive the drilling part for a reaming process (step S36). To be noted, the structural features and operation details of the above elements are all described in the above embodiments, so the descriptions thereof will be omitted.

Since the locating part of the guiding element can indicate the calculated drilling position, such as the pedicle, the guiding element of the invention can provide more precise drilling positioning. Moreover, the present invention further uses the auxiliary element and the k-pin to minimize the error range of the center of the pedicle. This can sufficiently improve the conventional error of guiding element positioning during the drilling/reaming operation.

In summary, the guiding element and the guiding assembly for spinal drilling operation of the present invention is composed of several elements, including the guiding element, auxiliary element, k-pin and cannular driller, so that the stability and precision of the reaming procedure during the spinal drilling operation can be enhanced.

Moreover, the specific functions of the above elements can help the spinal pedicle drilling operation. Regarding to the guiding element, since it has simple structure and is suitable for customization, the demand of the patient can be satisfied. Besides, the guiding element has a portion fitting the vertebra, so that the contact between the guiding element and the vertebra can be further improved. In detail, the clamping part has the hook portion which faces or bends toward the spinous process. One side of the hook portion in contact against the vertebra has a curvature corresponding to the surface of the spinous process, so that the guiding element can be disposed at the spinal process and across the vertebra through the hook portions. Accordingly, the contact between the guiding element and the vertebra can be further improved. In addition, the auxiliary element and the cannular driller are configured cooperating with the locating part. The auxiliary element can assist the location of the k-pin and prevent the non-stability in the conventional hand-hold procedure. The cannular driller can perform the reaming process through the locating part, thereby increasing the precision and decreasing the safety of spinal drilling operation.

Compared with the conventional art, the present invention still remains the conventional advantages of customizable and easy operation. In addition, based on its structural characteristics, the guiding element can be firmly disposed at the vertebra just through the hook portions of the clamping parts or the integral part. In other words, the guiding element of the preferred embodiment can be firmly disposed at the vertebra and cross the spinous process just though two parts contacting with the vertebra. Therefore, the guiding element and the guiding assembly of the present invention can further provide higher location stability and precision due to the structural property of the guiding element. Moreover, the two connection parts of the guiding element extends away from each other and will leave sufficient space at the side of locating parts for operation during the drilling process when the guiding element is disposed on the vertebra. Also, the two locating parts are both dangling from the vertebra through each connection part. Accordingly, during the drilling process, the operator can observe the drilling situation and the location where the k-pin actually drills into the vertebra through the gap between the locating parts and the vertebra, and the precision of spinal drilling operation is therefore increased.

Furthermore, the guiding assembly for spinal drilling operation utilizes an auxiliary element for facilitating and adjusting the penetrating position of the k-pin. This configuration can precisely match the hole drilled by the cannular driller with the targeted pedicle, so that the medical staffs can precisely drill into the center of the pedicle of the targeted vertebra.

Preferably, the guiding element of the present invention can have customized design based on the surface angle of the targeted vertebra (e.g. cervical, thoracic or lumber vertebra) of the patient. Accordingly, when the clamping parts and hook portions are pressed so as to push the guiding element, the guiding element can be still fixed on the targeted vertebra firmly, thereby increasing the precision and safety of the spinal drilling operation.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A guiding element for spinal drilling operation, which is configured to be disposed at a vertebra, wherein the vertebrae comprises a lamina, and the guiding element comprises:
a main body having two stand portions and each of the stand portion comprises a clamping part;
two connection parts, and each of the connection parts comprises a first end and a second end, each second end connects with one of the stand portions, each connection part extends in a direction from each second end to each first end, and the two first ends extends away from each other; and
two locating parts, and each locating part connects to each stand portion through each connection part,
wherein each clamping part comprises two hook portions, and each clamping part faces toward each other, and the main body is configured to be disposed at the lamina of the vertebra through the hook portions of the clamping parts, and each side, which is configured to be in contact against the vertebra, of the hook portions has a curvature corresponding to the surface of the lamina, and the sides of the each two hook portions of the same clamping part are continuous to one side of the same clamping part being configured to be in contact against the vertebra, so that the main body is configured to be disposed at the lamina and across the vertebra through the hook portions.

2. The guiding element of claim 1, wherein the main body further has a hand-held portion connecting to the stand portion.

3. The guiding element of claim 2, wherein the hand-held portion has a through hole.

4. The guiding element of claim 1, wherein each connection part forms an angle with each stand portion, and the angle is larger than 90 degrees.

5. The guiding element of claim 1, wherein each locating part is dangling from the vertebra through each connection part.

6. The guiding element of claim 1, wherein the vertebra is a cervical vertebra, thoracic vertebra, or a lumbar vertebra.

7. The guiding element of claim 1, wherein the two clamping part extends toward and connects to each other and forms an integral part.

8. A guiding assembly for spinal drilling operation, comprising:
a guiding element, which is configured to be disposed at a vertebra, wherein the vertebrae comprises a lamina, and the guiding element comprises:
a main body having two stand portions and each of the stand portion comprises a clamping part;
two connection parts, and each of the connection parts comprises a first end and a second end, each second end connects with one of the stand portions, each connection part extends in a direction from each second end to each first end, and the two first ends extends away from each other; and
two locating parts, and each locating part connects to each stand portion through each connection part,
wherein each clamping part comprises two hook portions, and each clamping part faces toward each other, and the main body is configured to be disposed at the lamina of the vertebra through the hook portions of the clamping parts, and each side, which is configured to be in contact against the vertebra of the hook portions, has a curvature corresponding to the surface of the lamina, and the sides of the each two hook portions of the same clamping part are continuous to one side of the same clamping part being configured to be in contact against the vertebra, so that the main body is disposed at the spinal process and across the vertebra through the hook portions.
at least an auxiliary element mounted on one of the locating part;
at least a k-pin mounted on the auxiliary element; and
at least a cannular driller mounted on the k-pin and having a holding part and a drilling part.

9. The guiding assembly of claim 8, wherein the main body further has a hand-held portion connecting to the stand portion.

10. The guiding assembly of claim 9, wherein the hand-held portion has a through hole.

11. The guiding assembly of claim 8, wherein each connection part forms an angle with each stand portion, and the angle is larger than 90 degrees.

12. The guiding assembly of claim 8, wherein each locating part is dangling from the vertebra through each connection part.

13. The guiding assembly of claim 8, wherein the vertebra is a cervical vertebra, thoracic vertebra, or a lumbar vertebra.

14. The guiding assembly of claim 8, wherein the two clamping part extends toward and connects to each other and forms an integral part.

15. The guiding assembly of claim 8, wherein the auxiliary element has a through hole along a longitudinal direction, and the k-pin passes through the through hole in the step of mounting the k-pin on the auxiliary element.
